(19) United States
(12) Patent Application Publication
(10) Pub. No.: US 2015/0013827 A1
(43) Pub. Date: Jan. 15, 2015

(54) RESERVOIR OR MEDICATED MODULE FOR A DRUG DELIVERY SYSTEM AND METHOD OF AND ASSEMBLY FOR FILLING THE SAME

(71) Applicant: SANOFI SA, Vernier (CH)

(72) Inventor: Bernd Kühn, Frankfurt am Main (DE)

(21) Appl. No.: 14/364,239

(22) PCT Filed: Dec. 14, 2012
(86) PCT No.: PCT/EP2012/075663

§ 371 Date: Jun. 10, 2014

(30) Foreign Application Priority Data
Dec. 15, 2011 (EP) 11193703.3

(51) Int. Cl. A61J 1/14; A61M 5/24; B65B 3/00; A61M 5/142

(52) U.S. Cl. CPC A61J 1/14; A61M 5/14244; A61M 5/24; B65B 3/003
USPC 141/2; 604/257; 141/25

(57) ABSTRACT

A reservoir for a drug delivery system is provided, wherein the reservoir comprises a cavity, the cavity being filled with a dose of a fluid drug such that the dose of the drug occupies 95% or more of the cavity, wherein the dose volume is less than 5 ml. Furthermore, a method for filling a reservoir for a drug delivery system is provided.

### CROSS REFERENCE TO RELATED APPLICATIONS

The present application is a U.S. National Phase Application pursuant to 35 U.S.C. §371 of International Application No. PCT/EP2012/075663 filed Dec. 14, 2012, which claims priority to European Patent Application No. 11193703.3 filed Dec. 15, 2011. The entire disclosure contents of these applications are herewith incorporated by reference into the present application.

### TECHNICAL FIELD

This disclosure relates to a method for filling a reservoir for a drug delivery system, an assembly for filling a reservoir for a drug delivery system and a reservoir for a drug delivery system.

### BACKGROUND

In a drug delivery system, often, a bung within a cartridge containing a plurality of doses of a drug is displaced by a piston rod. Thereby, a dose of the drug is expelled from the cartridge.

A drug delivery system is described in document WO 2008/058666 A1, for example.

### SUMMARY

It is an object of the present disclosure to provide a method for filling a reservoir for an improved drug delivery system, e.g. a drug delivery system having increased user safety, and an associated assembly to perform the method. Furthermore, it is an object of the present disclosure to provide a reservoir for an improved drug delivery system.

This object may, inter alia, be achieved by the subject matter of the independent claims. Advantageous embodiments and refinements are the subject matter of the dependent claims. However, further advantageous concepts may be disclosed herein besides the ones which are claimed.

One aspect relates to a method for filling a reservoir for a drug delivery system. The drug delivery system may comprise a pen-type drug delivery system. Alternatively, the drug delivery system may comprise an infusion pump. The reservoir may be configured for holding a dose of a fluid drug, in particular a single dose of the fluid drug. The dose may comprise a predefined volume. In particular, the dose may comprise a volume of less than 5 ml, preferably less than 1 ml. The dose may also be a microdose. The dose may comprise a volume of less than 50 μl, such as 30 μl or even less. The method may comprise one or more of the following steps:

A) Providing the reservoir. The reservoir may be empty, i.e. no volume of fluid drug may be retained in the reservoir, yet. The reservoir may define a cavity. The cavity may comprise an irregular, e.g. a non-cylindrical shape. The cavity may comprise a funnel-shape, for example. The cavity may be shaped such that it distributes a flow of the drug within the reservoir. The cavity may comprise one or more channels. The whole dose of the drug may be retained in the cavity once filled into the reservoir.

B) Reducing the pressure, in particular the gas pressure, within the cavity defined in the reservoir. The pressure may be reduced such that the pressure within the cavity is 200 mbar or less, for example. Reducing the pressure may mean creating a vacuum.

C) Filling a volume of the fluid drug, e.g. 10% of the dose of the drug, 30% of the dose of the drug, half of the dose of the drug, almost all of the dose of the drug or the complete dose of the drug, into the cavity while the pressure is reduced. During this filling step, the reduced pressure may be maintained at a given constant value.

D) Finishing the filling of the fluid drug into the cavity of the reservoir such that, after the filling was finished, the dose of drug is retained in the cavity of the reservoir. During this final filling step, the reduced pressure may be still maintained at the given constant value. Alternatively, the pressure may be increased, e.g. to normal pressure, after having filled a volume of the drug into the cavity as described in step C) and before finishing the filling of the fluid drug, e.g. before filling the remaining volume of the drug into the cavity. Said increased pressure may be maintained at a given constant value while filling the remaining volume of the drug into the cavity.

The reservoir is characterized in being complex to perform standard filling operations. Standard filling operations are associated with filling of cylindrically shaped containers like vials, bottles, cartridges, syringes and the like. Further, standard filling operations for sterile pharmaceutical products are related to fill volumes of more than 0.5 ml volume for injections up to about 1000 ml volume or more for infusion solutions or irrigation liquids. In some cases, fill volumes are in the range of 0.2 ml to 0.5 ml which is achievable using special dosing equipment, e.g. to produce prefilled syringes.

The inner volume of the reservoir, in particular the volume of the cavity defined by the reservoir, may be very small. For example, the volume of the cavity may comprise less than 0.2 ml, preferably less than 0.1 ml or even less, e.g. like 20-50 μl. However, as an alternative, the volume of the reservoir cavity may also be between 2 ml and 5 ml. The volume may amount to 1 ml, 1.5 ml, 2 ml, 2.5 ml, 3 ml, 3.5 ml, 4 ml, or even 4.5 ml, for example. For such greater volumes as compared to the small volumes of 1 ml or less, the reservoir may be suitable for use in infusion pumps that are, for example, attached to the skin or worn at the belt with a catheter connection to a needle piercing the skin (e.g. portable insulin pumps).

Complexity in relation to the above described reservoir may mean that the reservoir comprises a complex inner structure. For example, the reservoir may comprise a flow distribution or flow-direction system. The flow distribution or flow-direction system may be arranged in the interior of the reservoir. The cavity may be defined by the complex inner structure of the reservoir, e.g. the flow distribution system, and by the inner walls of the reservoir. The flow distribution or flow-direction system may comprise one, two or more channels. The channels and the inner walls of the reservoir define the previously mentioned cavity. The channels may comprise a small dimension or width. The channels may be curved. Thus, the cavity may be difficult to fill as capillary forces, dead or narrow ends or concave shapes of the channels prevent a rapid entry and distribution of the fluid in the cavity. Further, surface properties like hydrophobicity of some plastic materials may retain air bubbles in corners of the cavity that are difficult to remove for complete filling.

Due to the filling of the reservoir under the reduced pressure, the inclusion of gas, e.g. air, within the cavity may be minimized or even prevented. Consequently, due to the reduced pressure in the cavity during the filling process, the reservoir may be filled with the drug solution such that the fluid may occupy 95% or even more, e.g. 96%, 97%, 98%, 99% or 100%, of the cavity after the filling operation was completed. Preferably, the free volume of the reservoir, which may be defined by the volume of the empty cavity, is filled completely with the fluid drug after the filling operation was completed. Thus, dose accuracy may be increased. In particular, the risk of delivering an underdose from the reservoir, which may have fatal consequences for a user, may be minimized or even prevented. Provision of a drug delivery system having increased user safety may be facilitated in this way.

A further aspect relates to an assembly for filling a reservoir for a drug delivery system. The assembly may comprise an apparatus adapted and arranged for filling the reservoir. The assembly may be operable to perform the method for filling a reservoir for a drug delivery system as described above. The assembly may comprise a reservoir. The reservoir may comprise the previously described complex inner structure. The reservoir may have a cavity. The cavity may be configured for holding a dose, preferably a single dose, of a fluid drug. The assembly may further comprise a pressure member. The pressure member may be adapted and arranged to create reduced pressure within the cavity of the reservoir for filling the reservoir with the liquid drug. The assembly may further comprise a dosing valve. The dosing valve may be arranged and configured to, preferably automatically, fill the liquid drug into the cavity. The assembly may be configured to perform the operation of filling the cavity of the reservoir with a volume of the liquid drug under reduced pressure within the cavity.

The cavity may comprise a small width or dimension. The cavity may comprise a complex shape, e.g. a non-cylindrical shape. The cavity may be configured for distributing a flow of the drug within the reservoir. The cavity may comprise a channel, preferably two, three or more channels, as described above. The channels or parts of the channels may be curved. The channels may be configured to establish a fluid communication between a proximal end and a distal end of the reservoir. The respective channel may comprise a maximum width and a minimum width. The maximum width of the channel may be between 8 mm and 15 mm, for example. The maximum width may amount to 10 mm, for example. The minimum width of the channel may be between 0.5 mm and 2 mm, for example. The minimum width may amount to 1 mm, for example. For example, the fluid channel may be narrowing from about 10 mm upstream to 1 mm downstream to form a funnel shape. The channel may end in a capillary channel, preferably a curved capillary channel. The capillary channel may form a dead end of the channel. In particular, the capillary channel may comprise a width too small for the liquid to be filled directly into the capillary channel under normal environmental conditions and by means of ordinary filling methods. The capillary channel may comprise a width between 0.5 mm and 1.5 mm. The width of the capillary channel may be 1 mm, for example. The capillary channel may comprise a length between 4 mm and 6 mm. Preferably, the length may amount to 5 mm. The total volume of the cavity may be between 45 μl and 55 μl, preferably 50 μl, for example.

Due to the previously described filling operation, in particular due to the reduced pressure within the cavity during the filling operation, the formation of gas inclusions by the fluid drug may be reduced. Even more, due to the reduced pressure within the cavity during the filling operation the fluid drug may be prevented to define a gas inclusion on account of its viscosity and/or its surface tension within the cavity. Accordingly, the cavity and, in particular its previously described dead end, may be fillable completely with the liquid drug without gas or air being included in the cavity.

As a gas inclusion leads to disturbance of the liquid flow pattern, dose accuracy of a drug delivery system comprising such a reservoir may be increased in absence of gas bubbles. As entrapped gas is compressible, this may also be a cause for missing dose accuracy. Further, dosing of a gas bubble instead of drug solution reduces dose accuracy. Avoiding gas bubbles in the cavity may help to facilitate provision of a drug delivery system with increased user safety.

A further aspect relates to a reservoir for a drug delivery system. The reservoir may comprise a complex inner structure, as described above. The reservoir may comprise a cavity. The empty cavity of the reservoir may form a free volume of the reservoir. The cavity may have a complex shape. The cavity may have an irregular shape, for example a non-cylindrical shape or a funnel-shape. The cavity may be shaped such that it distributes a flow of the drug within the reservoir. The cavity may comprise channels, as described above. The cavity may hold a dose, preferably a single dose, of a fluid drug. The dose volume may be less than 5 ml, preferably less than 1 ml. Preferably, the volume of the dose of the fluid drug is less than 50 pl or less than 30 μl. The reservoir may be filled with the drug such that the dose of the drug occupies 95% or more, e.g. 96%, 97%, 98%, 99% or 100%, of the free volume of the reservoir. The reservoir may be filled according to the method described above. The reservoir may be filled by means of the assembly described above. At least one septum, preferably two septa, may be provided for closing the cavity, in particular the filled cavity.

Preferably, no gas or air is included in the reservoir, in particular in the cavity which defines the free volume of the reservoir before drug is filled into the cavity. In other words, the free volume may be completely filled with the liquid drug. In this way, dose accuracy may be increased. Dispensing of underdoses from the reservoir, which may have fatal or even lethal consequences for the user, may be prevented. This facilitates provision of an improved drug delivery system.

According to a preferred embodiment, a method for filling a reservoir for a drug delivery system is provided, the reservoir being configured for holding a dose of a fluid drug. The method comprises the following steps:

A) providing the reservoir,

B) reducing the pressure within a cavity defined in the reservoir,

C) filling a volume of the fluid drug into the cavity while the pressure is reduced,

D) finishing the filling of the fluid drug into the cavity of the reservoir such that, after the filling was finished, the dose of drug is retained in the cavity of the reservoir.

According to a preferred embodiment, an assembly for filling a reservoir for a drug delivery system is provided, the assembly being operable to perform the method as described above, the assembly comprising a reservoir having a cavity which is configured for holding a dose of a fluid drug, a pressure member which is adapted and arranged to create reduced pressure within the cavity of the reservoir for filling the reservoir with the liquid drug, and a dosing valve configured to fill the liquid drug into the cavity. The assembly is configured to perform the operation of filling the cavity of the reservoir with a volume of the liquid drug under reduced pressure within the cavity.

According to a preferred embodiment, a reservoir for a drug delivery system is provided,

wherein the reservoir comprises a cavity, the cavity being filled with a dose of a fluid drug such that the dose of the drug occupies 95% or more of the cavity, wherein the dose volume is less than 5 ml.

Of course, features described above in connection with different aspects and embodiments may be combined with each other and with features described below.

### BRIEF DESCRIPTION OF THE FIGURES

Further features and refinements become apparent from the following description of the exemplary embodiments in connection with the accompanying figures.

FIG. 1 schematically shows a perspective side view of a drug delivery system,

FIG. 2 schematically shows a perspective side view of a part of the drug delivery system of FIG. 1,

FIG. 3 schematically shows a sectional side view of a part of the drug delivery system of FIG. 1,

FIG. 4 schematically shows a perspective view of an embodiment of a reservoir for a drug delivery system,

FIG. 5 schematically shows a sectional side view of an embodiment of a reservoir for a drug delivery system,

FIG. 6A schematically shows a perspective view of an inner structure of a reservoir for a drug delivery system,

FIG. 6B schematically shows a perspective view of a reservoir for a drug delivery system comprising the inner structure of FIG. 6A,

FIG. 6C schematically shows a perspective view of an inner structure of a reservoir for a drug delivery system,

FIG. 6D schematically shows a perspective view of a reservoir for a drug delivery system comprising the inner structure of FIG. 6C,

FIG. 7 schematically shows a perspective view of embodiments for an inner structure of a reservoir for a drug delivery system,

FIG. 8 schematically shows a perspective view of a reservoir for a drug delivery system,

FIG. 9 schematically shows a perspective view of a reservoir for a drug delivery system,

FIG. 10 schematically shows a perspective view of a reservoir for a drug delivery system,

FIG. 11 schematically shows an assembly for filling a reservoir for a drug delivery system,

FIG. 12 schematically shows a part of the assembly of FIG. 11,

FIG. 13 schematically shows a part of the assembly of FIG. 11,

FIG. 14 schematically shows a part of the assembly of FIG. 11.

Like elements, elements of the same kind and identically acting elements may be provided with the same reference numerals in the figures.

### DETAILED DESCRIPTION

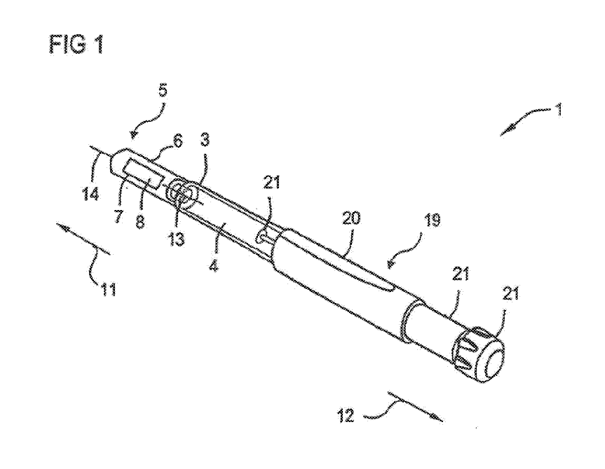

In FIGS. 1, 2 and 3 a drug delivery system 1 or parts of the drug delivery system 1 are shown. The drug delivery system 1 may comprise a pen-type injection system as it is shown in FIGS. 1 to 3 and as it is described in connection with the FIGS. 1 to 14. As an alternative (not explicitly shown in the Figures), the drug delivery system may comprise an infusion pump that is, for example, attached to the skin (patch pump) or worn at a belt with a catheter connection to a needle piercing the skin (e.g. portable insulin pump).

In the embodiment shown in the Figures, the drug delivery system 1 comprises a drug delivery device 19. The drug delivery system 1 comprises a medicated module 5. The medicated module 5 is, preferably releasably, connected to the drug delivery device 19, e.g. by a threaded connection, to form the drug delivery system 1. The drug delivery device 19 may be a stand-alone device, i.e. a device which is configured to perform a dose setting and dose delivery operation without the medicated module 5 being connected thereto. The drug delivery device 19 may be operable for performing a plurality of dose setting and dose delivery operations. The medicated module 5 may be configured to operate in combination with the drug delivery device 19, preferably only. The medicated module 5 may be operable for performing a single dose delivery operation.

The drug delivery device 19 comprises a housing 20. The medicated module 5 comprises a housing 6. The drug delivery system 1 and/or a component thereof have a distal end and a proximal end. The distal end is indicated by arrow 11. The proximal end is indicated by arrow 12. The term “distal end” designates that end of the drug delivery system 1 or a component thereof which is or is to be arranged closest to a dispensing end of the drug delivery system 1. The term “proximal end” designates that end of the system 1 or a component thereof which is or is to be arranged furthest away from the dispensing end of the system 1. The distal end and the proximal end are spaced apart from one another in the direction of an axis. The axis may be the longitudinal axis of the drug delivery system 1, or elements thereof like the medicated module 5 or the drug delivery device 19.

In the following, the components of the drug delivery device 19 and the medicated module 5 are explained in detail:

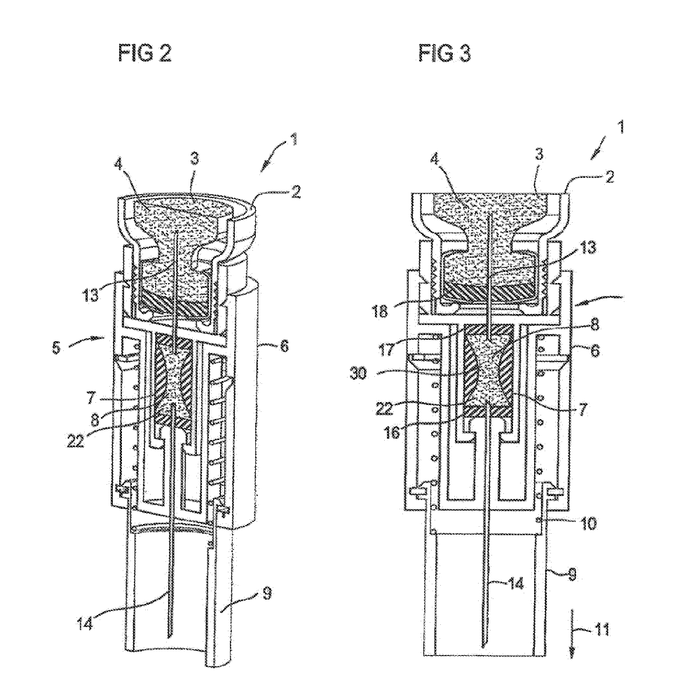

The drug delivery device 19 comprises a reservoir, in particular a primary reservoir 3. The primary reservoir 3 may be retained within a reservoir holder 2 (see, for example, FIGS. 2 and 3). The reservoir holder 2 stabilizes the position of the primary reservoir 3 mechanically. The reservoir holder 2, in particular the proximal end of the reservoir holder 2, is connectable, e.g. by a threaded engagement, by a weld or by a snap-fit, to the housing 20 of the drug delivery device 19. The reservoir holder 2, in particular the distal end of the reservoir holder 2, is connectable, e.g. by a threaded engagement, by a weld or by a snap-fit, to the housing 6 of the medicated module 5. In an alternative embodiment (see FIG. 1), the primary reservoir 3 may be directly connected to the housing 6 of the medicated module 5 and to the housing 20 of the drug delivery device 19. In this case, the reservoir holder 2 may be redundant.

The primary reservoir 3 contains a primary drug 4, preferably a plurality of doses of the primary drug 4. The primary drug 4 may be a liquid drug. The term “drug”, as used herein, preferably means a pharmaceutical formulation containing at least one pharmaceutically active compound,

wherein in one embodiment the pharmaceutically active compound has a molecular weight up to 1500 Da and/or is a peptide, a proteine, a polysaccharide, a vaccine, a DNA, a RNA, an enzyme, an antihousing or a fragment thereof, a hormone or an oligonucleotide, or a mixture of the above-mentioned pharmaceutically active compound,

wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis,

wherein in a further embodiment the pharmaceutically active compound comprises at least one peptide for the treatment and/or prophylaxis of diabetes mellitus or complications associated with diabetes mellitus such as diabetic retinopathy,

wherein in a further embodiment the pharmaceutically active compound comprises at least one human insulin or a human insulin analogue or derivative, glucagon-like peptide (GLP-1) or an analogue or derivative thereof, or exendin-3 or exendin-4 or an analogue or derivative of exendin-3 or exendin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivates are for example B29-N-myristoyl-des(B30) human insulin; B29-N-palmitoyl-des (B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-Y-glutamyl)-des(B30) human insulin; B29-N-(N-lithocholyl-Y-glutamyl)-des(B30) human insulin; B29-N-(ω-carboxyheptadecanoyl)-des(B30) human insulin and B29-N-(w-carboxyhepta-decanoyl) human insulin.

Exendin-4 for example means Exendin-4(1-39), a peptide of the sequence H His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH2.

Exendin-4 derivatives are for example selected from the following list of compounds:

### H-(Lys)4-des Pro36, des Pro37 Exendin-4(1-39)-NH2,

### H-(Lys)5-des Pro36, des Pro37 Exendin-4(1-39)-NH2,

### des Pro36 Exendin-4(1-39),

### des Pro36 [Asp28] Exendin-4(1-39),

### des Pro36 [IsoAsp28] Exendin-4(1-39),

### des Pro36 [Met(O)14, Asp28] Exendin-4(1-39),

### des Pro36 [Met(O)14, IsoAsp28] Exendin-4(1-39),

### des Pro36 [Trp(O2)25, Asp28] Exendin-4(1-39),

### des Pro36 [Trp(O2)25, IsoAsp28] Exendin-4(1-39),

### des Pro36 [Met(O)14 Trp(O2)25, Asp28] Exendin-4(1-39),

### des Pro36 [Met(O)14 Trp(O2)25, IsoAsp28] Exendin-4(1-39); or

### des Pro36 [Asp28] Exendin-4(1-39),

### des Pro36 [IsoAsp28] Exendin-4(1-39),

### des Pro36 [Met(O)14, Asp28] Exendin-4(1-39),

### des Pro36 [Met(O)14, IsoAsp28] Exendin-4(1-39),

### des Pro36 [Trp(O2)25, Asp28] Exendin-4(1-39),

### des Pro36 [Trp(O2)25, IsoAsp28] Exendin-4(1-39),

### des Pro36 [Met(O)14 Trp(O2)25, Asp28] Exendin-4(1-39),

### des Pro36 [Met(O)14 Trp(O2)25, IsoAsp28] Exendin-4(1-39),

wherein the group -Lys6-NH2 may be bound to the C-terminus of the Exendin-4 derivative;
or an Exendin-4 derivative of the sequence

### des Pro36 Exendin-4(1-39)-Lys6-NH2 (AVE0010),

### H-(Lys)6-des Pro36 [Asp28] Exendin-4(1-39)-Lys6-NH2,

### des Asp28 Pro36, Pro37, Pro38Exendin-4(1-39)-NH2,

### H-(Lys)6-des Pro36, Pro38 [Asp28] Exendin-4(1-39)-NH2,

### H-Asn-(Glu)5des Pro36, Pro37, Pro38 [Asp28] Exendin-4(1-39)-NH2,

### des Pro36, Pro37, Pro38 [Asp28] Exendin-4(1-39)-(Lys)6-NH2,

### H-(Lys)6-des Pro36, Pro37, Pro38 [Asp28] Exendin-4(1-39)-(Lys)6-NH2,

### H-Asn-(Glu)5-des Pro36, Pro37, Pro38 [Asp28] Exendin-4(1-39)-(Lys)6-NH2,

### H-(Lys)6-des Pro36 [Trp(O2)25, Asp28] Exendin-4(1-39)-Lys6-NH2,

### H-des Asp28 Pro36, Pro37, Pro38 [Trp(O2)25] Exendin-4(1-39)-NH2,

### H-(Lys)6-des Pro36, Pro37, Pro38 [Trp(O2)25, Asp28] Exendin-4(1-39)-NH2,

### H-Asn-(Glu)5-des Pro36, Pro37, Pro38 [Trp(O2)25, Asp28] Exendin-4(1-39)-NH2,

### des Pro36, Pro37, Pro38 [Trp(O2)25, Asp28] Exendin-4(1-39)-(Lys)6-NH2,

### H-(Lys)6-des Pro36, Pro37, Pro38 [Trp(O2)25, Asp28] Exendin-4(1-39)-(Lys)6-NH2,

### H-Asn-(Glu)5-des Pro36, Pro37, Pro38 [Trp(O2)25, Asp28] Exendin-4(1-39)-(Lys)6-NH2,

### H-(Lys)6-des Pro36 [Met(O)14, Asp28] Exendin-4(1-39)-Lys6-NH2,

### des Met(O)14 Asp28 Pro36, Pro37, Pro38 Exendin-4(1-39)-NH2,

H-(Lys)6-desPro36, Pro37, Pro38 [Met(O)14, Asp28] Exendin-4(1-39)-NH2,

### H-Asn-(Glu)5-des Pro36, Pro37, Pro38 [Met(O)14, Asp28] Exendin-4(1-39)-NH2,

### des Pro36, Pro37, Pro38 [Met(O)14, Asp28] Exendin-4(1-39)-(Lys)6-NH2,

### H-(Lys)6-des Pro36, Pro37, Pro38 [Met(O)14, Asp28] Exendin-4(1-39)-(Lys)6-NH2,

### H-Asn-(Glu)5 des Pro36, Pro37, Pro38 [Met(O)14, Asp28] Exendin-4(1-39)-(Lys)6-NH2,

### H-Lys6-des Pro36 [Met(O)14, Trp(O2)25, Asp28] Exendin-4(1-39)-Lys6-NH2,

### H-des Asp28 Pro36, Pro37, Pro38 [Met(O)14, Trp(O2)25] Exendin-4(1-39)-NH2,

### H-(Lys)6-des Pro36, Pro37, Pro38 [Met(O)14, Asp28] Exendin-4(1-39)-NH2,

### H-Asn-(Glu)5-des Pro36, Pro37, Pro38 [Met(O)14, Trp(O2)25, Asp28] Exendin-4(1-39)-NH2,

### des Pro36, Pro37, Pro38 [Met(O)14, Trp(O2)25, Asp28] Exendin-4(1-39)-(Lys)6-NH2,

### H-(Lys)6-des Pro36, Pro37, Pro38 [Met(O)14, Trp(O2)25, Asp28] Exendin-4(S1-39)-(Lys)6-NH2,

### H-Asn-(Glu)5-des Pro36, Pro37, Pro38 [Met(O)14, Trp(O2)25, Asp28] Exendin-4(1-39)-(Lys)6-NH2;

or a pharmaceutically acceptable salt or solvate of any one of the afore-mentioned Exendin-4 derivative.

Hormones are for example hypophysis hormones or hypothalamus hormones or regulatory active peptides and their antagonists as listed in Rote Liste, ed. 2008, Chapter 50, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

A polysaccharide is for example a glucosaminoglycane, a hyaluronic acid, a heparin, a low molecular weight heparin or an ultra low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Antibodies are globular plasma proteins (−150 kDa) that are also known as immunoglobulins which share a basic structure. As they have sugar chains added to amino acid residues, they are glycoproteins. The basic functional unit of each antihousing is an immunoglobulin (Ig) monomer (containing only one Ig unit); secreted antibodies can also be dimeric with two Ig units as with IgA, tetrameric with four Ig units like teleost fish IgM, or pentameric with five Ig units, like mammalian IgM.

The Ig monomer is a “Y”-shaped molecule that consists of four polypeptide chains; two identical heavy chains and two identical light chains connected by disulfide bonds between cysteine residues. Each heavy chain is about 440 amino acids long; each light chain is about 220 amino acids long. Heavy and light chains each contain intrachain disulfide bonds which stabilize their folding. Each chain is composed of structural domains called Ig domains. These domains contain about 70-110 amino acids and are classified into different categories (for example, variable or V, and constant or C) according to their size and function. They have a characteristic immunoglobulin fold in which two β sheets create a “sandwich” shape, held together by interactions between conserved cysteines and other charged amino acids.

There are five types of mammalian Ig heavy chain denoted by α, δ, ε, γ, and μ. The type of heavy chain present defines the isotype of antihousing; these chains are found in IgA, IgD, IgE, IgG, and IgM antibodies, respectively.

Distinct heavy chains differ in size and composition; α and γ contain approximately 450 amino acids and 6 approximately 500 amino acids, while μ and ε have approximately 550 amino acids. Each heavy chain has two regions, the constant region (CH) and the variable region (VH). In one species, the constant region is essentially identical in all antibodies of the same isotype, but differs in antibodies of different isotypes. Heavy chains γ, α and δ have a constant region composed of three tandem Ig domains, and a hinge region for added flexibility; heavy chains μ and ε have a constant region composed of four immunoglobulin domains. The variable region of the heavy chain differs in antibodies produced by different B cells, but is the same for all antibodies produced by a single B cell or B cell clone. The variable region of each heavy chain is approximately 110 amino acids long and is composed of a single Ig domain.

In mammals, there are two types of immunoglobulin light chain denoted by λ and κ. A light chain has two successive domains: one constant domain (CL) and one variable domain (VL). The approximate length of a light chain is 211 to 217 amino acids. Each antihousing contains two light chains that are always identical; only one type of light chain, κ or λ, is present per antihousing in mammals.

Although the general structure of all antibodies is very similar, the unique property of a given antihousing is determined by the variable (V) regions, as detailed above. More specifically, variable loops, three each the light (VL) and three on the heavy (VH) chain, are responsible for binding to the antigen, i.e. for its antigen specificity. These loops are referred to as the Complementarity Determining Regions (CDRs). Because CDRs from both VH and VL domains contribute to the antigen-binding site, it is the combination of the heavy and the light chains, and not either alone, that determines the final antigen specificity.

An “antihousing fragment” contains at least one antigen binding fragment as defined above, and exhibits essentially the same function and specificity as the complete antihousing of which the fragment is derived from. Limited proteolytic digestion with papain cleaves the Ig prototype into three fragments. Two identical amino terminal fragments, each containing one entire L chain and about half an H chain, are the antigen binding fragments (Fab). The third fragment, similar in size but containing the carboxyl terminal half of both heavy chains with their interchain disulfide bond, is the crystalizable fragment (Fc). The Fc contains carbohydrates, complement-binding, and FcR-binding sites. Limited pepsin digestion yields a single F(ab′)2 fragment containing both Fab pieces and the hinge region, including the H-H interchain disulfide bond. F(ab′)2 is divalent for antigen binding. The disulfide bond of F(ab′)2 may be cleaved in order to obtain Fab'. Moreover, the variable regions of the heavy and light chains can be fused together to form a single chain variable fragment (scFv).

Pharmaceutically acceptable salts are for example acid addition salts and basic salts. Acid addition salts are e.g. HCl or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1 C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology.

Pharmaceutically acceptable solvates are for example hydrates.

A bung (not explicitly shown in the Figures) is moveably retained within the primary reservoir 3. The bung seals the primary reservoir 3 proximally. Movement of the bung in the distal direction with respect to the primary reservoir 3 causes the primary drug 4 to be dispensed from the primary reservoir 3, provided that fluid communication between the distal end of the primary reservoir 3 and the environment, e.g. via a needle, is established as described below in detail.

The drug delivery device 19 may be a pen-type device, in particular a pen-type injector. The device 19 may be configured for dispensing fixed doses of the primary drug 4, i.e. doses which may not be varied by a user, or variable doses of the primary drug 4. The device 19 can be a re-usable device, which means that the primary reservoir 3 can be replaced, in particular during a reset operation, by a replacement reservoir for dispensing a plurality of doses from the replacement reservoir. Alternatively, the device 19 may be a disposable device. In this case, the primary reservoir 3 is non-releasably connected to the reservoir holder 2. The drug delivery device 19 may be a multidose device, i.e. a device configured for setting and dispensing a plurality of doses of the primary drug 4. The drug delivery device 19 comprises a drive mechanism 21. The drive mechanism 21 is used for setting and dispensing a dose of the primary drug 4.

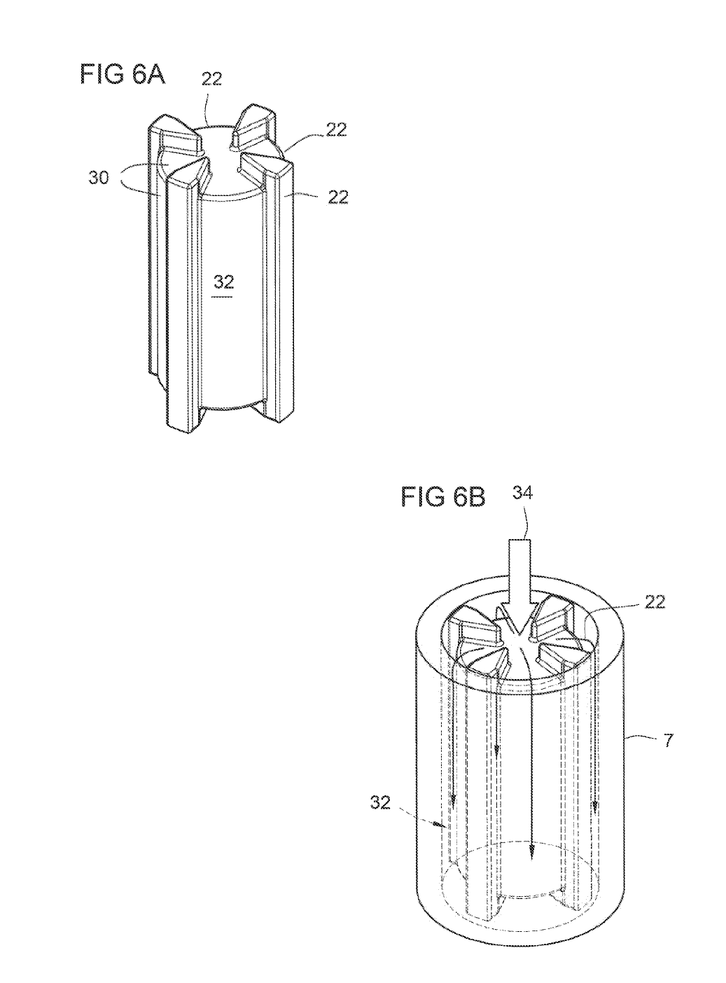

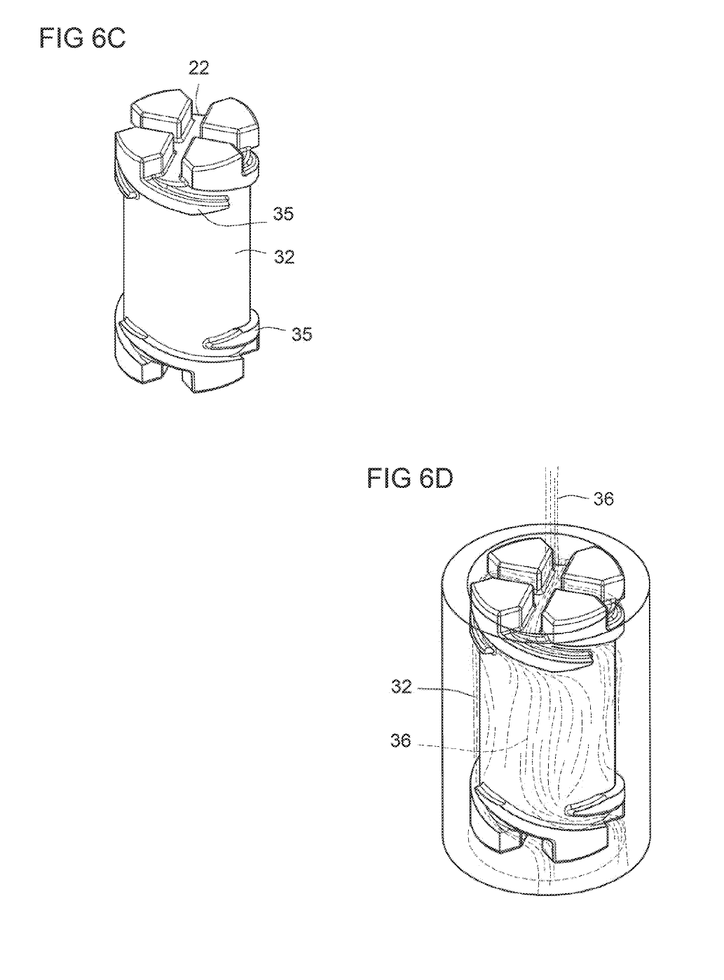

The medicated module 5 comprises a secondary reservoir 7. The reservoir 7 is a so-called primary package (PP). The secondary reservoir 7 may be made from plastics, for example. The secondary reservoir 7 comprises a cavity 30 (see, for example FIG. 6A). The cavity 30 holds a fluid secondary drug 8, preferably a single dose of the fluid secondary drug 8. The dose of the fluid secondary drug 8 is a fixed dose, i.e. a dose which cannot be varied by the user. The volume of the fluid secondary drug 8 is less than 5 ml, for example 1 ml. The dose may be a microdose, preferably a single microdose, of the secondary drug 8. Preferably, the volume of the fluid secondary drug 8 is less than 100 μl. Preferably, the volume of the fluid secondary drug 8 is greater than 1 μl. The volume of the fluid secondary drug 8 amounts to 25 μl, for example. As an alternative, when the secondary reservoir 7 is provided in an infusion pump, the volume of the drug 8 may amount to values greater than 1 ml, e.g. 4 ml or 4.5 ml. When the reservoir 7 is provided for use with an infusion pump, the medicated module as well as the primary reservoir may be redundant and the secondary reservoir 7 may be integrated directly as a single reservoir in the infusion pump.

The secondary drug 8 comprises a GLP-1 and/or insulin, for example. The medicated module 5 may be a one-use device, i.e. a device which is used only once, in particular for dispensing the fixed dose of the secondary drug 8.

The medicated module 5 comprises a first septum 16 (see FIG. 3). The first septum 16 is arranged in the distal end section of the secondary reservoir 7. The first septum 16 seals the secondary reservoir 7 distally. The medicated module 5 comprises a second septum 17 (see FIG. 3). The second septum 17 is arranged in the proximal end section of the reservoir 7. The second septum 17 seals the secondary reservoir 7 proximally. The primary reservoir 3 comprises a septum 18 (see FIG. 3). The septum 18 is arranged in the distal end section of the primary reservoir 3. The septum 18 seals the primary reservoir 3 distally.

The medicated module 5 comprises a first needle 14. The first needle 14 is provided in the distal end section of the medicated module 5. The medicated module 5 comprises a second needle 13. The second needle 13 is provided in the proximal end section of the medicated module 5. The secondary reservoir 7 is configured for fluid connection with the first and second needle 13, 14. The first needle 14, in particular the proximal end of the first needle 14, pierces the first septum 16 of the secondary reservoir 7 for establishing fluid connection with the secondary reservoir 7. The first needle 14, in particular the distal end of the first needle 14, is configured for piercing the skin of the user. The second needle 13, in particular the distal end of the second needle 13, pierces the second septum 16 of the secondary reservoir 7 for establishing fluid connection with the secondary reservoir 7. The second needle 13, in particular the proximal end of the second needle 13, pierces the septum 18 of the primary reservoir 3 for establishing fluid connection with the primary reservoir 3.

The medicated module 5 comprises a needle guard 9 (see FIGS. 2 and 3). The needle guard 9 is used for protecting the first needle 14 against environmental influences. The needle guard 9 is automatically forced in the distal direction with respect to the housing 6 by means of a spring 10 (see FIGS. 2 and 3).

The drug delivery system 1 is configured for dispensing the fixed dose of the fluid secondary drug 8 from the secondary reservoir 7 along with a, preferably variable, dose of the primary drug 4 from the primary reservoir 3. In particular, the drug delivery system 1 is configured for dispensing two separate drugs 4, 8 from separate reservoirs 3, 7 without previously mixing the drugs 4, 8. Thereby, at first, a dose of the primary drug 4 contained in the primary reservoir 3 is set by the user by operating the drive mechanism 21. In a next step, the user further operates the drive mechanism 21 such that the set dose of the primary drug 4 is moved from the primary reservoir 3 in the distal direction with respect to the housing 20. The set dose of the primary drug 4 is moved from the primary reservoir 3 via the second needle 13 and into the secondary reservoir 7. Thereby, the set dose of the primary drug 4 forces the non-user settable dose of the secondary drug 8 from the secondary reservoir 7, in particular out of the cavity 30. A mixing of the set dose of the primary drug 4 with the fixed dose of the fluid secondary drug 8 from the secondary reservoir 7 is prevented by means of an inner structure of the secondary reservoir 7, which is described later on in detail. Upon completion of the delivery procedure, preferably the complete volume of the secondary drug 8 of the secondary reservoir 7, i.e. the single dose of the secondary drug 8, has been expelled as well as the set dose of the primary drug 4 of the primary reservoir through the first needle 14.

The secondary reservoir 7 comprises an inner structure, in particular a complex inner structure. According to the shown embodiments, the inner structure comprises a flow distributor 32 (see, for example FIGS. 5, 6A to 6D, 7, 8, 9 and 10). The flow distributor 32 may be made from plastics. The flow distributor 32 may be an insert of the secondary reservoir 7. Alternatively, the flow distributor 32 may be integrally formed with the secondary reservoir 7. The combination of the inner structure, i.e. the flow distributor 32, and the reservoir 7, in particular the inner walls of the reservoir 7, defines the cavity 30 of the secondary reservoir 7. The flow distributor 32 is located in the interior of the secondary reservoir 7 as illustrated in FIG. 6B, for example, to minimize the residual volume of the secondary drug 8 that is caused by recirculation and/or stagnant zones and that might remain in secondary reservoir 7 at the end of the dose delivery operation.

Preferably, the flow distributor 32 is designed such that at least about 80% of the fluid volume of the secondary drug 8 is expelled from the secondary reservoir 7 through the first needle 14. Most preferably, at least about 90% should be expelled. Ideally, displacement of the primary drug 4 from the drug delivery device 19 into the second needle 13 will displace the complete volume of the secondary drug 8 without substantial mixing of the two drugs 4, 8. In particular, the flow distributor 32 is used to minimize the risk of mixing occurring between the two drugs 4, 8 during dispense, therefore promoting plug flow.

The flow distributor 32 comprises at least one channel 22 (see, for example FIGS. 2 and 3). Preferably, the flow distributor 32 comprises two, three or more channels 22 (see, for example, FIG. 6A). The respective channel 22 extends between the distal end and the proximal end of the reservoir 7. The respective channel 22 establishes a fluid communication between the distal end and the proximal end. The respective channel is shaped such that it distributes a flow of the drug 8 within or through the reservoir 7. The channels 22 define, together with the inner walls of the reservoir 7, the cavity 30 of the secondary reservoir 7.

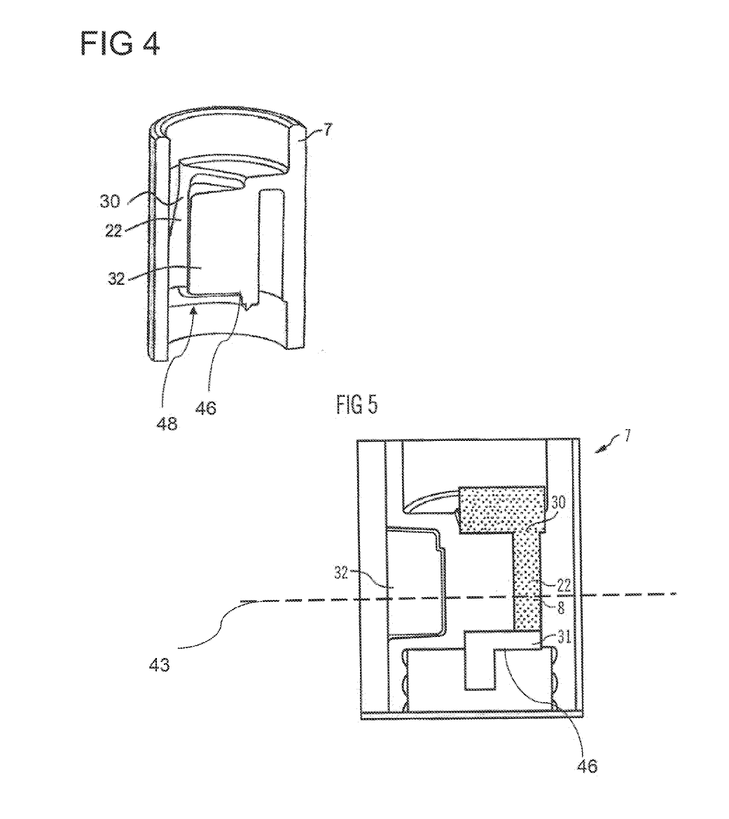

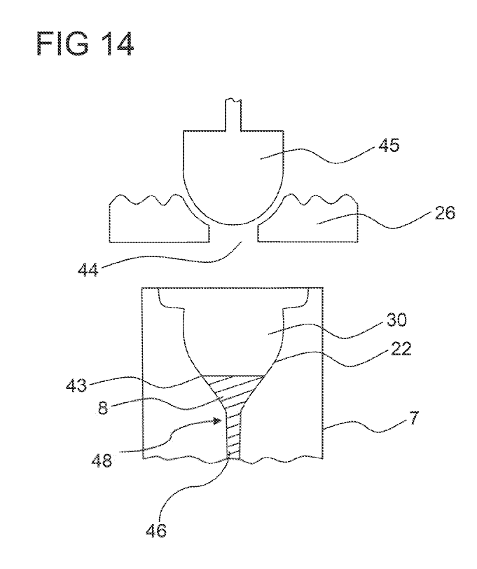

The respective channel 22 comprises a maximum diameter or width and a minimum diameter or width, which can be gathered from FIGS. 4 and 5, for example. The maximum diameter or with is between 8 mm and 15 mm, for example. The maximum diameter amounts to 10 mm, for example. The minimum diameter or with is between 0.5 mm and 2 mm, for example. The minimum diameter amounts to 1 mm, for example. For example, the respective channel 22 narrows from about 10 to 1 mm in the direction of the needle 14 (see FIG. 4) to form a funnel shape (see FIG. 14). The channel 22 may be curved as can be seen, for example, in FIGS. 4 and 5. The channel 22 terminates in a capillary channel 48 (see FIG. 14), preferably a curved capillary channel. The capillary channel 48 comprises a small width or diameter. In other words, the capillary channel 48 is narrow. In particular, the capillary channel 48 may comprise a dimension too small for the drug 8 to be filled directly into the capillary channel 48 under normal environmental conditions and by means of ordinary filling methods. The capillary channel 48 forms a so-called dead end of the channel 22. The capillary channel 48 comprises a diameter or width between 0.5 mm and 1.5 mm. The diameter or width of the capillary channel 48 may be 1 mm, for example. The capillary channel 48 comprises a length between 4 mm and 6 mm. Preferably, the length amounts to 5 mm. Due to this complex structure of the respective channel 22, the cavity 30, which is defined by the channel 22 and the inner walls of the reservoir 7, comprises a complex shape. In particular, the shape of the cavity 30 is irregular, e.g. non-cylindrical. The cavity 30 may be funnel-shaped.

The empty cavity 30 defines the free volume of the reservoir 7. The volume of the cavity 30 is less than 5000 ml, preferably less than 1000 mm³. Most preferred, the volume of the cavity 30 is less than 100 mm³. The volume of the cavity 30 may be greater than 1 mm³. The volume of the cavity 30 amounts to 25 mm³, for example. The secondary drug 8 is retained in the cavity 30 defined by the channel 22.

Two possible embodiments of the flow distributor 32 are illustrated in FIGS. 6A, 6B, 6C and 6D. According to said figures, the flow distributor 32 comprises a cylindrical pin or insert. According to the embodiment of FIGS. 6A and 6B, the flow distributor 32 is positioned in the secondary reservoir 7 and is configured such that the secondary drug 8 fills four channels 22, which are defined by the shape and location of two or more support ribs 33. In a preferred embodiment, the flow distributor 32 is located so that the edges of the respective flow channel 22 are in direct contact with the inner walls of the reservoir 7 (FIG. 6B). The flow distributor 32 can be constructed of any material that is compatible to the primary and secondary drugs 4, 8. A preferred material is one that is typically used to manufacture septa or pistons (bungs) found in multi-dose medicament cartridges, however, any other material that is compatible with the drug 4, 8 could be used, e.g. glass, plastics or specific polymers. The shape of the flow channels 22 can be optimized to promote plug flow of medicament, shown by arrow 34 in FIG. 6B, by varying the dimensions, geometry and number of support ribs 33. The cross-sectional area of the annulus formed between the flow distributor 32 and the wall of the reservoir 7 should be kept relatively small. The free volume available to store the secondary drug 8 would equal the internal volume of the secondary reservoir 7 minus the volume of the flow distributor 32. Therefore, if the volume of the flow distributor 32 is marginally smaller than the internal volume of the reservoir 7, a small volume is left which the secondary drug 8 occupies. Hence, the scale of both the reservoir 7 and the flow distributor 32 can be large while storing a small volume of medicament. As such, the external geometry of the secondary reservoir 7 is not dictated by the volume of secondary drug 8. As a result, for small volumes of secondary drug 8 (e.g. 5 to 500 μl) the medicated module 5 can be of an acceptable size for handling, transport, manufacture and assembly.

FIGS. 6C and 6D show another embodiment of the flow distributor 32 that includes one or more radial vanes 35. In addition, the width of the channels 22 present in the radial flow portion are narrowed to accelerate the flow at the base to help expel the secondary drug 8 and to reduce the stagnant volume present on the first septum 16. Additionally, radial features like these may also help reduce/eliminate drug loss during the filling process due to splashing as the flow distributor 32 is inserted. In essence, they would help acting like baffle plates to deflect/contain any upward splashing that occurs. FIG. 6D shows the drug flow 36 through the secondary reservoir 7 obtained from computational fluid dynamic modelling. When using the flow distributor 32 shown in FIGS. 6C and 6D, the computer modelling predicts that less than 5% residual volume of the secondary drug 8 will remain in the secondary reservoir 7 after dose delivery, thus achieving about 95% expulsion.

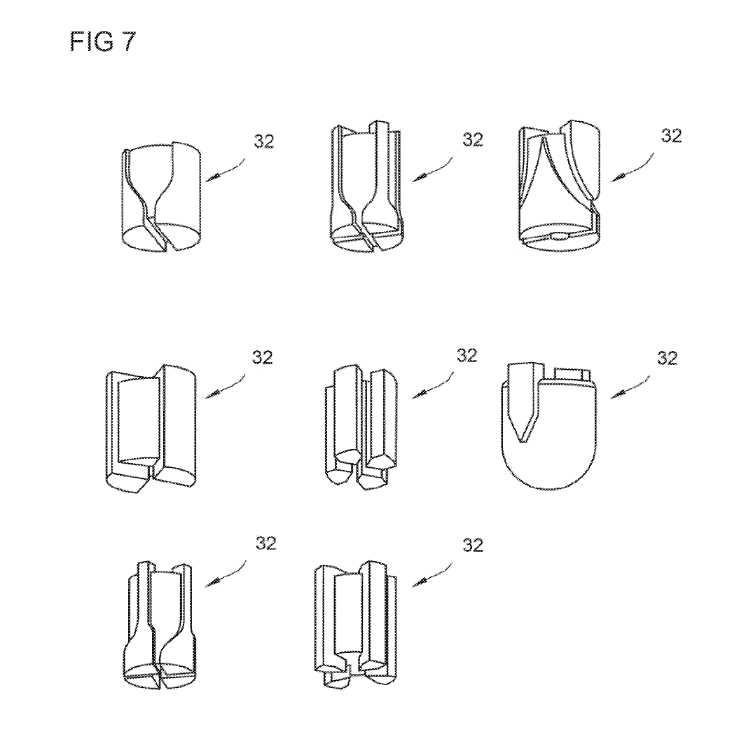

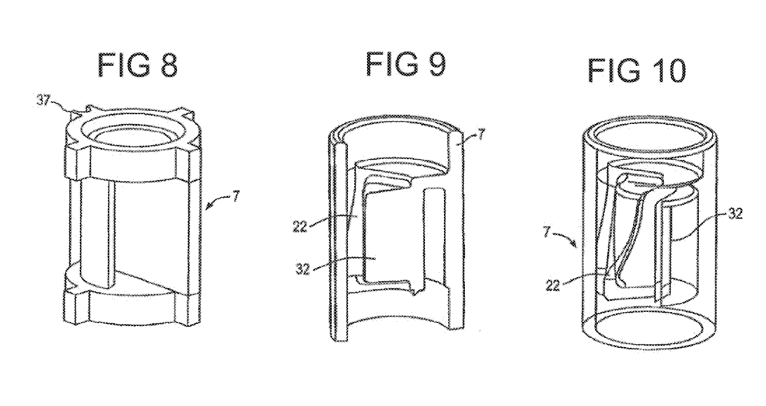

FIG. 7 illustrates alternative embodiments of the flow distributor 32. The embodiments show different shapes, in particular widths, for the respective channel 22 of the flow distributor 32. Of course, the flow distributor 32 could equally comprise a combination of structural components within the medicated module 5. For example, the inner surface of the secondary reservoir 7 could be configured with grooves and ribs to define fluid flow channels and the septae 16, 17 could be configured to assist in changing fluid flow from axial to radial to swirl or vice versa. Likewise, various parts of the flow distributor 32 could be in-moulded during the fabrication of the secondary reservoir 7 and, preferably, manufactured from the same plastic material as the reservoir wall, however, other materials suitable for two- or multi-component injection moulding could be used. An exemplary embodiment is shown in FIGS. 8 and 9 where a flow distributor 32 is in-moulded with the wall of the secondary reservoir 7.

The medicated module 5 can also include a bypass channel 37 as shown in FIG. 8 that is incorporated as part of secondary reservoir 7 to facilitate priming of the first needle 14 with the primary drug 4.

The width of the respective channel 22, which defines the cavity 30, may be so small such that the fluid drug 8 can on account of its surface tension and/or viscosity under normal environmental conditions, e.g. under normal pressure of 1013 mbar, define a gas inclusion, e.g. an inclusion of air, within the channel 22, in particular within the capillary channel 48, when the secondary reservoir 7 is filled with the secondary drug 8 under normal conditions. The gas inclusion (see gas inclusion 31 in FIG. 5, for example) could extend over the whole width of the channel 22. In other words, the width of the channel 22, in particular of the capillary channel 48 (see FIGS. 4 and 14) is so small that the filling of the secondary reservoir 7 could, under normal environmental conditions, lead to the inclusion of an air bubble within the channel 22. In this way, it could occur that not the complete volume of the drug 18 is filled into the channel 22 and/or that air is dispensed along with the drug 8, which might lead to underdosing. Moreover, an air inclusion could obstruct the drug flow in the cavity 30, which may lead to turbulences of the primary and the secondary drug 4, 8 during the delivery operation. This may interrupt the previously described plug flow which may lead to dose inaccuracies, as well.

The volume of potentially entrapped air is dependent on a number of parameters. Firstly, surface tension and viscosity of the filled drug 8 may determine to what extent the solution can penetrate into the capillary structure, e.g. the capillary channel 48, of a given diameter having a dead end. Secondly, the boiling point of the filled drug 8 determines the level of vacuum that can be applied to the system at filling. If for example the boiling point (i.e. transformation of liquid phase to gas phase) of the liquid drug 8 at a filling temperature of 20° C. is reached at 50 mbar pressure, this will be the lower limit of the vacuum setting for the filling process. To avoid boiling and, thus, gas formation of the liquid during filling, the pressure limit for the filling process may be set at 100 mbar, for example, to include a safety margin. After the filling process is completed and the pressure is back to ambient pressure of about 1000 mbar, the drug 8 in the cavity is forced under this increased pressure into the dead ends, in particular into the capillary channel 48. If e.g. the entrapped air in a capillary channel 48 of 1 mm diameter and 5 mm length at 100 mbar pressure has a volume of 3.9 μl, this may be reduced by factor 10 when the pressure raises from 100 mbar to 1000 mbar, i.e. to 0.39 μl. If the total volume of the filled reservoir 7 is 50 μl, the reduced air volume of 0.39 μl in the capillary channel 48 may be about 0.78% of the reservoir volume. In this example the effective fill volume may amount to 99.2%. By optimization of formulation parameters boiling point, surface tension and/or viscosity, as well as geometric dimensions of capillary channels 48 in the cavity 30 and applied pressure during filling, the reservoir 7 can be filled with minimum to no residual air volume.

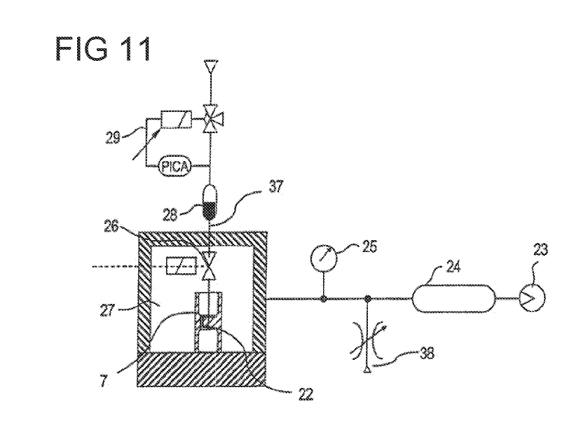

For avoiding the inclusion of air and, hence, underdosing, the secondary reservoir 7 is filled in the following way (see in particular FIGS. 11, 12 and 13):

1) In a first step, an assembly, e.g. an apparatus, for filling the reservoir 7 is provided. The assembly is configured to perform the operation of filling the cavity 30 of the secondary reservoir 7 with the liquid drug 8 under reduced pressure within the cavity 30.

Thereby, at first, the previously described secondary reservoir 7 is provided. The reservoir 7 is empty. In particular, the cavity 30 is, of course, not yet filled with the liquid drug 8.

Furthermore, a pressure member 23 is provided. The pressure member 23 is adapted and arranged to create a vacuum within the cavity 30. In particular, the pressure member 23 is adapted and arranged to create reduced pressure within the cavity 30 of the reservoir 7 for filling the reservoir 7 with the liquid drug 8. The pressure member 23 may be a vacuum pump. The pressure member 23 may be a rotary slide valve vacuum pump. The pressure member 23 may be the rotary slide valve vacuum pump VACFOX VC75 or the vacuum pump M24 of the manufacturer Vacuubrand, for example.

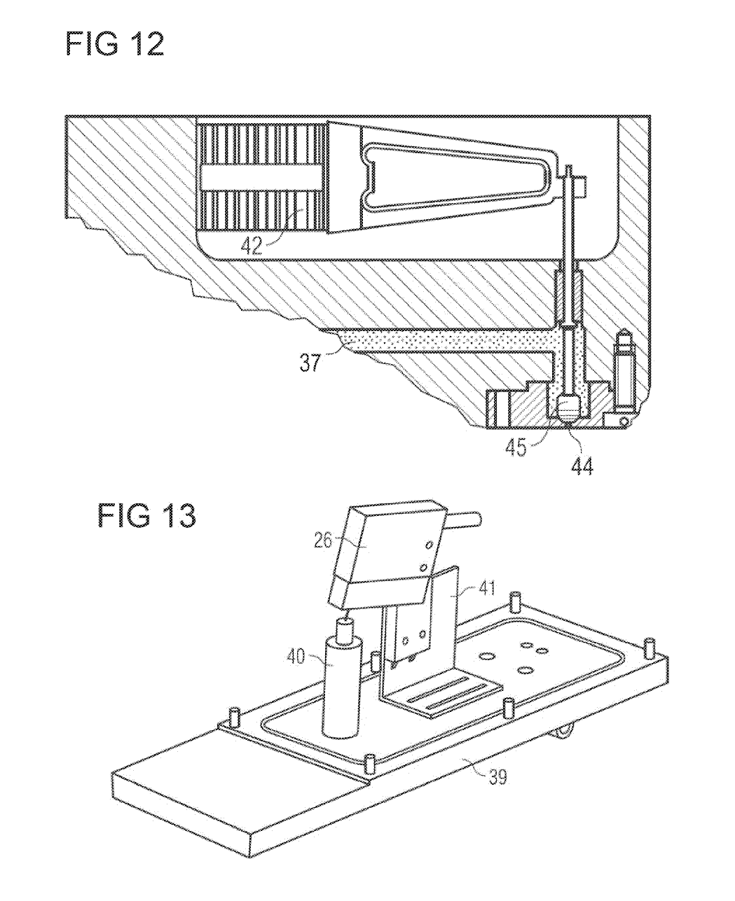

Furthermore, a dosing valve 26 is provided (see, in particular, FIG. 12). The dosing valve 26 is configured to fill the liquid drug 8 into the cavity 30. The dosing valve 26 is a piezo valve. The dosing valve comprises an outlet 44. The liquid drug 8 is dispended from the outlet 44 of the dosing valve 26 into the cavity 30 during the filling operation.

Furthermore, a pressurized reservoir 28 is provided. The reservoir 28 comprises the fluid drug 8. Preferably, the reservoir 28 comprises a volume of the fluid drug 8 which is greater the dose volume of the fluid drug which is to be filled via the dosing valve 26 into the cavity 30. The fluid drug 8 is moved from the reservoir 28 into the dosing valve 26 via a delivery channel 37. The pressure for supplying the drug 8 from the pressurized reservoir 28 is controlled via a control valve 29.

Inside the dosing valve 26 (see FIG. 12), there is a plunger with a ceramic seal ball 45 (see also FIG. 14). The plunger can be moved in an axial direction via a piezo package 42 of the dosing valve 25. When the plunger has reached its maximum displacement in the direction of the outlet 44, the dosing valve 26 is closed. This piezo drive is able to realize very short switching times, which is described later on in more detail.

The dosing valve 26 enables a time-pressure dependent filling of the cavity 30 with the drug 8. The dosing valve 26 is configured to fill the cavity 30 under a predetermined frequency, e.g. 1000 Hz, with which the outlet 44 of the dosing valve 26 is repeatedly opened and closed. In other words, the dosing valve 26 enables a high-frequent opening and closing of the outlet 44. In this way, tiny droplets of the drug 8 with a volume of less than 1 μl, preferably 10 nl, may emerge from the outlet 44 to be filled into the cavity 30. This has the particular advantage that the volume of the respective droplets is small enough such that it does not define a gas inclusion within the channel 22 as described above.

The frequency of the dosing valve 26 may be chosen such that a potential impact on the vacuum within the cavity 30 and within the fluid path of the drug 8 in the direction towards the cavity 30 may be minimized. In particular, the opening times of the dosing valve 26 may be minimized to reduce the impact on the vacuum. Moreover, the predetermined frequency of the dosing valve 26 is chosen such that the drug 8 can disperse within the cavity 30, in particular within the channel 22, before the outlet 44 is opened the next time. The slower the valve closing speed, the fewer splashes are generated. However, the minimum speed is limited by the tendency of drop formation on the outlet 44. For example, the outlet 44 is opened for only 10 μs is before it is closed again. This has the advantage that, once the fluid drug 8 has emerged from the outlet 44, it can freely flow into the cavity 30, thereby dispersing into the cavity 30.

The dosing valve 26 is, in addition, equipped with an integrated temperature control. With the temperature control, the drug 8 to be filled can be heated up to 180° C., if necessary. This function holds the temperature of the drug 8 constant during the filling operation, thus preventing variations of the fill quantity of the drug 8 that would be caused by temperature variations. The temperature control also supports the sterilization of the dosing valve 26 with hot steam. The temperature can be adjusted to the viscosity of the drug 8 to be filled into the cavity 30. Accordingly, due to the temperature control, drugs with very different viscosities can be filled into the cavity 30.

An electronic control of the dosing valve 26 may further be provided. By means of the electronic control of the dosing valve 26, the operation of filling the reservoir 7 can be adjusted to the characteristics of different drugs 8 to be filled into the cavity 30. An electronic feedback for controlling the filling operation may further be provided.

In contrast to other systems for filling the liquid drug 8 into the cavity 30 under a reduced pressure, e.g. a needle, the dosing valve 26 has the advantage that the volume of the drug 8 which is to be filled into the cavity 30 can be accurately controlled. In other words, the dosing valve 26 enables a high dosing accuracy. In particular, if the drug 8 was filled into the cavity 30 by means of a needle, the fluid drug 8 could flow out of the needle in an uncontrollable way and the reduced pressure in the cavity 30 could influence the head of liquid drug 8 in the needle and the delivery channel 37.

Furthermore, for filling small given volumes of liquid drug 8 into the cavity 30, a needle with a tiny inner diameter would be needed. Thereby, the fluid drug 8 would on account of its surface tension adapt the shape of a droplet once having emerged from the needle. The diameter of the droplet could thereby be greater than the lumen of the needle. If this droplet entered the cavity 30, i.e. the channel 22, it could block the channel 22, thereby including air 31 retained in the lower parts of the channel 22 (see FIG. 5). Hence, filling the reservoir 7 with ordinary systems such as a needle would lead to increased air inclusions in the cavity 30, which would negatively affect the dose accuracy of the drug delivery system 1 as described above.

In contrast thereto, the dosing valve 26 can be adapted, e.g. the diameter of the dosing valve 26, can be chosen such that the droplets of the fluid drug 8 passing from the outlet 44 comprise only a tiny diameter as described above. Said diameter can be adjusted to the diameter of the cavity 30, in particular to the width of the channel 22. In this way, the drug 8 can deeply disperse into the volume of the cavity 30. Air inclusions, which may arise from large droplets of the drug 8 obstructing the channel 22 can be avoided in this way.

As last part of the assembly for filling the reservoir 7, a vacuum chamber 27 is provided. The reservoir 7 is put into the vacuum chamber 27. Also, the dosing valve 26 is put into the vacuum chamber 27. The dosing valve 26 is connected to a base plate 39 via an adjustable dosing valve holder 41 (see FIG. 13). The reservoir 7 is mounted under the dosing valve 26 via a holder 40 (FIG. 13).

2) In a next step, the pressure within the cavity 30 of the reservoir 7 is reduced. This is achieved by the pressure member 23 (see FIG. 11). In particular, the pressure member 23 evacuates the vacuum chamber 27 wherein the reservoir 7 is retained. The reduced pressure can be read out via a vacuum sensor 25. The pressure can be adjusted via an adjustable throttle 38. Via the throttle 38, the quantity of excess air that enters the system can be adjusted. That means, the more excess air enters, the higher the absolute pressure that must be reached in the vacuum chamber 27.

The reduction of the pressure within the cavity 30 has the advantage that the fluid drug 8 can still disperse better within the cavity 30, in particular within the complex structure of the cavity 30, during the filling operation as creation of very tiny droplets of the fluid drug 8 is enhanced which droplets can be filled into the small cavity 30 without creating inclusions of gas or air.

The reduction of the pressure within the cavity 30 further has the advantage that the inclusion of air in the cavity 30, in particular in the dead narrow ends of the capillary channels 48 (see FIGS. 4 and 14) during the filling operation is further minimized or even avoided. In particular, air which is included in the capillary channel 48 during the filling operation is reduced in dependence from the absolute pressure established in the empty volume, i.e. the cavity 30, during the filling operation such that only a minimum residual volume of included air or even no more air is present after the filling operation was completed.

The reduced pressure should be as low as possible. The reduced pressure within the cavity 30 is expediently less than 200 mbar. Preferably, the reduced pressure is greater than 50 mbar. For example, the reduced pressure amounts to 100 mbar. The reduced pressure is chosen in dependence from the vapour pressure of the liquid drug 8 to be filled into the cavity 30. In particular, the reduced pressure is chosen such that it is greater than the vapour pressure of the liquid drug 8. The reduced pressure amounts preferably to a value which is about four times the value of the vapour pressure of water at a temperature of about 20° C. This is advisable for avoiding spontaneous vaporization of the liquid drug 8 under the reduced pressure.

3) In a next step of the filling procedure, a volume of the fluid drug 8 is filled into the cavity 30 while the pressure is reduced.

There are two embodiments for filling the drug 8 into the cavity 30. According to both embodiments, the pressure within the cavity 30 is reduced and then, kept constant, over a given time.

In the first embodiment, the reservoir 7 is filled such that a first volume of the dose is filled under a first pressure within the cavity 30 and such that the remaining volume of the dose is filled under a second pressure within the cavity 30. Thereby, the second pressure is greater than the first pressure. The first pressure may be the reduced pressure, e.g. 100 mbar. The second pressure may be normal pressure, e.g. 1013 mbar. The first pressure and the second pressure comprise a constant value over a given time. In particular, the drug 8 is filled into the cavity 30 under the reduced pressure until the drug 8 has reached a predetermined filling level 43, as shown in FIG. 14. The predetermined filling level 43 may be above the dead-ended narrow structures 46 of the cavity 30. In particular, the predetermined filling level 43 may be above the capillary channel 48. As shown in FIG. 5, the drug 8 may block the narrow structure 46 of the capillary channel 48 such that air may be enclosed in the capillary channel 48. The enclosed air 31 may be removed during the further filling operation. The enclosed air 31 may be forced, in particular compressed, into the structure 46 during further filling operation. In this way, the structure 46 of the capillary channel 48 may no longer be completely blocked by the air 31. Accordingly, the capillary channel 48 will be available for being filled with the drug 8.

When the drug 8 has reached the predetermined filling level 43, the further volume of the drug 8 still to be filled into the cavity 30, e.g. droplets of the remaining drug 8, directly impinges onto the surface of the volume of drug 8 which was already filled into the cavity 30. This may lead to turbulences in the fluid drug 8 and, furthermore, ambient air may be included in the fluid drug 8 by means of the droplets impinging onto the surface. It may be possible that, during further filling of the drug 8 under reduced pressure, this may lead to an enhanced formation of gas bubbles in the fluid drug 8 as compared to the filling under normal pressure. Said gas bubbles could degas upon inclusion of further air in the fluid drug 8. This may increase the volume of the fluid drug 8 within the cavity 30. In the worst case, this could lead to the drug 8 overflowing the cavity 30. The volume of drug 8 overflowing the cavity 30 can no longer be dispensed from the cavity 30 during a delivery operation. Thus, an underdose may be delivered to the user. In order to avoid this scenario, the pressure may be adjusted during the filling operation. In particular, when the drug 8 has reached the predetermined filling level 43, the pressure within the cavity 30 is increased. In particular, when the drug 8 has reached the predetermined filling level 43, the pressure is increased until normal pressure is established within the cavity 30. Afterwards the cavity 30 is filled with the remaining volume of the drug 8 until the dose of drug 8 is retained in the reservoir 7. The ambient pressure in the cavity 30 thereby helps to prevent the gas bubbles in the fluid drug 8 from degassing.

In the second embodiment, the total volume of the drug 8, i.e. the whole dose of the drug 8, is filled into the complex inner structures of the cavity 30, i.e. the channels 22 with the dead-ended narrow structures given by the capillary channels 48, while the pressure is reduced, in particular while the pressure is kept at the reduced value. This means that, when the drug 8 has reached the predetermined filling level 43 (FIG. 14), the reduced pressure is maintained within the cavity 30 and the cavity 30 is filled with the remaining volume of the drug 8 until the dose of drug 8 is retained in the reservoir 7. The reduced pressure comprises a constant value over the total filling time. After the cavity 30 was filled with the fluid drug 8, the reduced pressure is increased to normal pressure. In this embodiment, prior to filling the drug 8 into the cavity 30, the fluid drug 8 may be exposed to reduced pressure, e.g. vacuum, for avoiding gas bubbles within the fluid drug 8. Moreover, the temperature within the cavity 30 can be chosen such that gas bubbles in the fluid drug 8 are prevented from degassing.

In both embodiments, the volume of air which may be included during the filling operation, may be reduced by a factor of 10 when the pressure is increased from the reduced pressure (e.g. 100 mbar) to the normal pressure (e.g. 1000 mbar) after the filling operation was completed (second embodiment) or after the first volume of the drug 8 was filled into the cavity 30 (first embodiment). When the pressure is increased, the drug 8 is forced under this increased pressure into the dead ended narrow structures 46 of the cavity 30, in particular into the capillary channels 48. In this way, potentially enclosed air 31 is removed from the cavity 30.

When the filling operation is completed, the reservoir 7 is filled with the drug 8 such that the dose of the drug 8 occupies 95% or more of the volume of the cavity 30. The dose of the drug 8 may occupy 96%, 97%, or 98% of the volume of the cavity 30. The dose of the drug 8 may occupy 99% of the volume of the cavity 30. The dose of the drug 8 may occupy 100% of the volume of the cavity 30.

The filling of the cavity 30 under reduced pressure and by means of the dosing valve 26 has in particular the advantage that a fully automatic filling operation under reduced pressure or under vacuum with a high filling precision is enabled. Furthermore, by means of the previously described method and/or assembly, doses with a volume in the range of nm to ml can be filled into the cavity 30.

When the filling operation is completed, the reservoir 7 is closed with the septa 16, 17 as described in connection with FIGS. 1 to 3.

FIG. 4 shows an embodiment of the secondary reservoir 7.

The secondary reservoir 7 is filled with the single dose of the liquid drug 8 after having completed the filling operation as described above. In particular, the drug 8 is held in the channel 22 which is defined by the flow distributor 32 and the reservoir 7. Preferably, no air is included in the channel 22.

Now, the reservoir 7 is ready to be inserted into the medicated module 5 and, then, the medicated module 5 can be releasably connected to the drug delivery device 19 for dispensing the combination of the primary and the secondary drugs 4, 8 with high dose accuracy and user safety. Alternatively, the reservoir 7 may be inserted as a single drug reservoir into an infusion pump (not explicitly shown) for dispensing the content of the reservoir 7 to a user.

1-22. (canceled)

23. A method for filling a reservoir for a drug delivery system, the reservoir being configured for holding a dose of a fluid drug, wherein the method comprises the following steps,
a) providing the reservoir;
b) reducing the pressure within a cavity defined in the reservoir;
d) filling a volume of the fluid drug into the cavity while the pressure is reduced; and
d) finishing the filling of the fluid drug into the cavity of the reservoir such that, after the filling was finished, the dose of drug is retained in the cavity of the reservoir.

24. The method of claim 23, wherein the reduced pressure within the cavity is less than 200 mbar, and wherein the volume of the dose of drug is less than 5 ml.

25. The method according to claim 24, wherein the reservoir is filled with the drug such that the dose of the drug occupies 95% or more of the empty cavity.

26. The method according to claim 23, wherein the drug is filled into the cavity by means of a dosing valve, wherein the dosing valve is a piezo valve, and wherein the dosing valve comprises an outlet, wherein the drug is dispensed from the outlet of the dosing valve into the cavity when the dosing valve is open, and wherein the dosing valve is repeatedly opened and closed while filling the drug into the cavity.

27. The method according to claim 26, wherein the volume of the drug that emerges from the outlet when the dosing valve is open, is less than 1 μl, preferably 10 nl.

28. The method according to claim 26, wherein the opening and closing frequency is chosen such that the drug can disperse within the cavity before the outlet is opened the next time.

29. The method according to claim 26, wherein the dosing valve is opened and closed with a frequency within a range from 100 Hz to 1000 Hz.

30. The method according to claim 26, wherein the dosing valve is equipped with an integrated temperature control and wherein the temperature of the drug is held constant during filling.

31. The method according to claim 23, wherein the drug is filled into the cavity under the reduced pressure until the drug has reached a predetermined filling level, wherein the predetermined filling level is defined by the filling level with respect to the reservoir being above a structure of the cavity, wherein the structure is so small that it may be blocked by entrapped air during the filling operation.

32. The method according to claim 31, wherein the cavity terminates in a capillary channel having a structure, wherein the structure is so small such that it may be blocked by entrapped air in the capillary channel.

33. The method of claim 31, further comprising when the drug has reached the predetermined filling level, increasing the pressure within the cavity and, then, filling the cavity with the remaining volume of the drug until the dose of drug is retained in the reservoir.

34. The method of claim 31, further comprising when the drug has reached the predetermined filling level, maintaining the reduced pressure within the cavity and filling the cavity with a further volume of the drug until the dose of drug has been filled into the reservoir.

35. The method according to claim 31, wherein, when the dose of drug has been filled into the reservoir, the entrapped air is forced into the structure of the cavity.

36. The method according to claim 33 in combination with claim 32, wherein, when the dose of drug has been filled into the reservoir, the drug is forced into the capillary channel under increased pressure.

37. A reservoir for a drug delivery system, wherein the reservoir comprises a cavity, the cavity being filled with a dose of a fluid drug such that the dose of the drug occupies 95% or more of the cavity, wherein the dose volume is less than 5 ml.

38. The reservoir according to claim 37, wherein the cavity has a non-cylindrical shape.

39. The reservoir according to claim 37, wherein the cavity is shaped such that it distributes a flow of the drug within the reservoir.

40. The reservoir according to claim 37, wherein the cavity comprises at least one channel, and wherein the reservoir comprises a distal end and a proximal end, wherein the channel is configured to establish a fluid communication between the distal end and the proximal end of the reservoir.

41. The reservoir according to claim 40, wherein the channel has a width which is so small such that the fluid drug on account of its surface tension and/or its viscosity can define a gas inclusion within the channel if it was attempted to fill the fluid drug into the channel under normal environmental conditions.

42. The reservoir according to claim 37, wherein the volume of the cavity is less than 1 ml.

43. The reservoir according to claim 37, wherein the reservoir comprises a flow distributor, and wherein the flow distributor and an inner wall of the reservoir in combination define the cavity.

44. An assembly for filling a reservoir for a drug delivery system, the assembly being operable to perform the method according to claim 23, the assembly comprising
a reservoir having a cavity which is configured for holding a dose of a fluid drug,
a pressure member which is adapted and arranged to create reduced pressure within the cavity of the reservoir for filling the reservoir with the liquid drug, and
a dosing valve configured to fill the liquid drug into the cavity,
wherein the assembly is configured to perform the operation of filling the cavity of the reservoir with a volume of the liquid drug under reduced pressure within the cavity.
